(19) United States
(12) Patent Application Publication
(10) Pub. No.: US 2016/0085941 A1
(43) Pub. Date: Mar. 24, 2016

(54) Method of Discovery of Effective Components in Herbals Based on Evidences by Reversed-directed Analysis

(71) Applicant: Jianyi Zhang, Clarksville, MD (US)

(72) Inventor: Jianyi Zhang, Clarksville, MD (US)

(21) Appl. No.: 14/866,924

(22) Filed: Sep. 26, 2015

Related U.S. Application Data
(60) Provisional application No. 62/177,349, filed on Mar. 13, 2015.

(51) Int. Cl. G06F 19/00; A61B 5/021; A61B 5/00; G06F 19/24

(52) U.S. Cl. CPC G06F 19/3475; G06F 19/24; G06F 19/322; G06F 19/363; G06F 19/366; A61B 5/021; A61B 5/4848

(57) ABSTRACT

A method of proving safety and efficacy of herbal compounds by clinical information obtained by physicians in daily practice by implementation of network technology, chemical analysis, statistics, and clinical trials. Data for potential therapeutic compounds are directly obtained from human being in daily practice setting, application of the invention will tremendously reduce the time and costs involved in research and development of drugs.

### CROSS-REFERENCE TO RELATED APPLICATIONS

The present application relates and claims priority to U.S. provisional patent application No. 62/177,349 filed on Mar. 13, 2015.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

1. Field of the Invention

The present invention relates to discover effective and safe components in herbal medication.

2. Description of the Related Art

Development and application of chemical compounds to treat illness is a hallmark of modern medicine. Western medicine is a product of modern chemistry, research and development, which would take an average of 15 years from research to market, and costs about 1.5 billion US dollars on average. Due to high cost, only one unknown compound can be tested to prove its safety and efficacy. Study of two or more unknown compounds in the same trial would cost much more, which few pharmaceutical companies can bear with it. Traditional Chinese Medicine (TCM) has made an indelible contribution for health of the Chinese people. TCM is a natural medicine; its effect must have its inherent material basis, which might be achieved by a single or multiple chemical components. Herbals have very complex chemical compositions, even a single herb consists of hundreds unknown compounds, and TCM is composed with several or even dozens of herbals, safety and efficacy might be involved with different compounds from various herbals and their metabolites. Classical method to study TCM is to extract a single chemical constituents from an herbal, then analyze the activity of the ingredients one by one, in vivo or in vitro. However, the method cannot detect safety and efficacy of metabolites, nor synergistic effects among different compounds in TMC. There are numerous methods to analyze chemical compounds in herbals, such as high pressure liquid chromatography (HPLC), gas chromatography—mass spectrometry, liquid chromatography—mass spectrometry, liquid chromatography—mass—mass spectrometry (LCMS-MS), fingerprints, and so on. However, these methods can only identify peaks corresponding to their chemical compositions, cannot associate them with effectiveness and safety. Therefore, the material basis is still bottleneck in TMC study.

### BRIEF SUMMARY OF THE INVENTION

The present invention is a method to identify the active ingredients of traditional Chinese medicine, the present invention include a website to allow providers to record patients' condition, diagnosis and treatment through the website. The patients' data can be collected by provider to conduct statistical analysis. The chemical compositions of herbals can be analyzed. By the present invention provider can conduct both internal and external tests to determine the active ingredients, and treat patients with pure effective compounds or together with combination of herbals.

### DESCRIPTION OF THE DRAWINGS

For a more complete understanding of the present invention, reference is now made to the following descriptions:

FIG. 1 Digitize TMC which provide information for safety and efficacy of herbals.

FIG. 2 Analyze and determine effective components.

FIG. 3 Selection Common Components among Patients with Preferred Outcome.

FIG. 4 Discovery of effective anti-bacterial components.

FIG. 5 Chemical analysis shows the highest concentration of W, Y, and Z in each patient.

FIG. 6 Patient's response and their genetic types.

FIG. 7 Classification of patients based on their response to herbals.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

Internet and website are mature modern technologies. To achieve the goal, the first thing is to establish a website specialized for TMC doctors to collect general and clinical information in their practice, information such as name, gender, age, nationality, place of birth, symptoms and signs, diagnosis, family, surgical history, medications, test results, imaging and pathology results, side-effects are collected. Information in website should be downloadable with permission; each user can analyze those data. Practitioner will be taught to how to use the program and conduct statistical analysis.

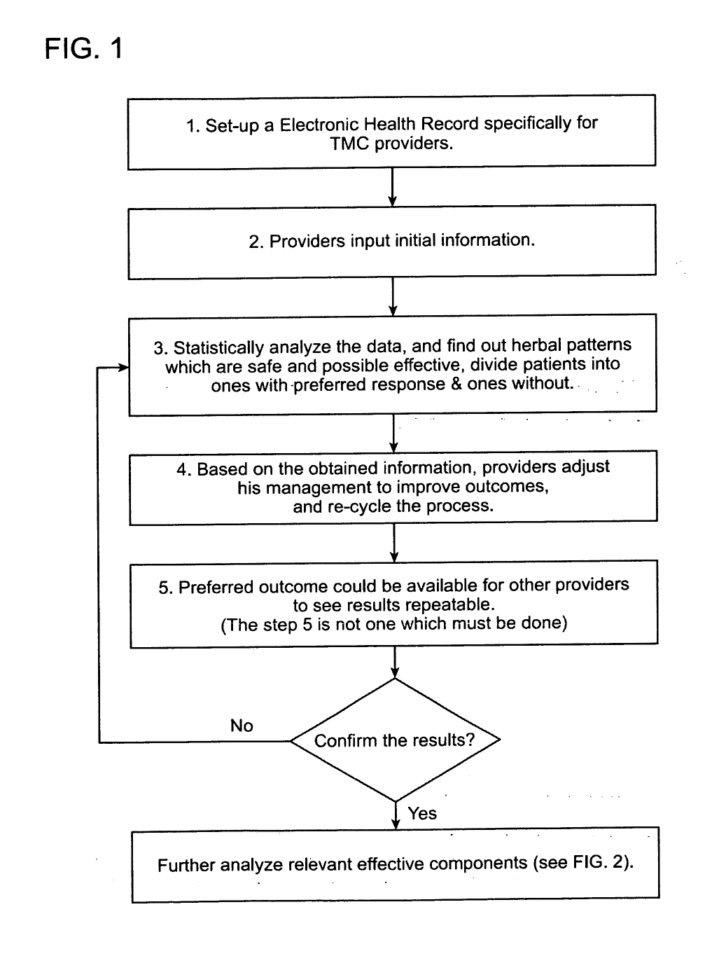

A same concoction may have different results in various patients, through whom the concoction could be found safe and effective, or safe, but not effect. Doctors can analyze the results, and adjust compositions, re-treat patients to improve outcome. Practitioners can compare patients with different treatments to detect side effects of herbals. Preferred results could be announced or published, other doctors can repeat these results in the same way, which makes TMC measurable and repeatable, and transform it from experience to science. If preferred results are found, the next step is the analysis and discovery of active ingredients, the process is illustrated in FIG. 1.

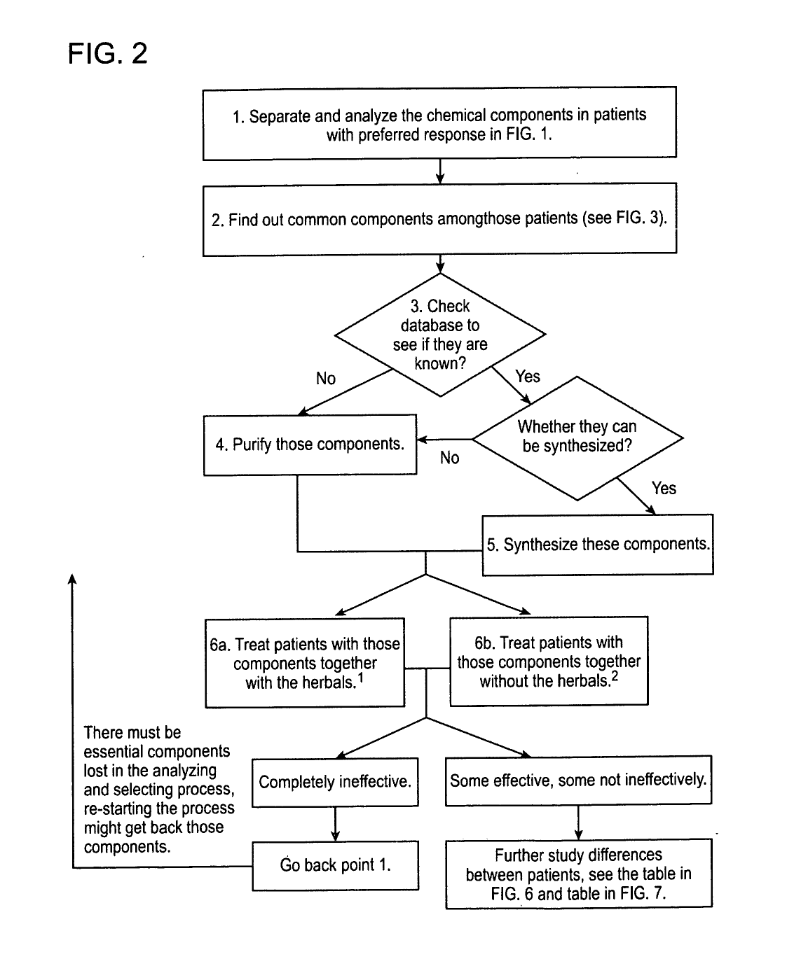

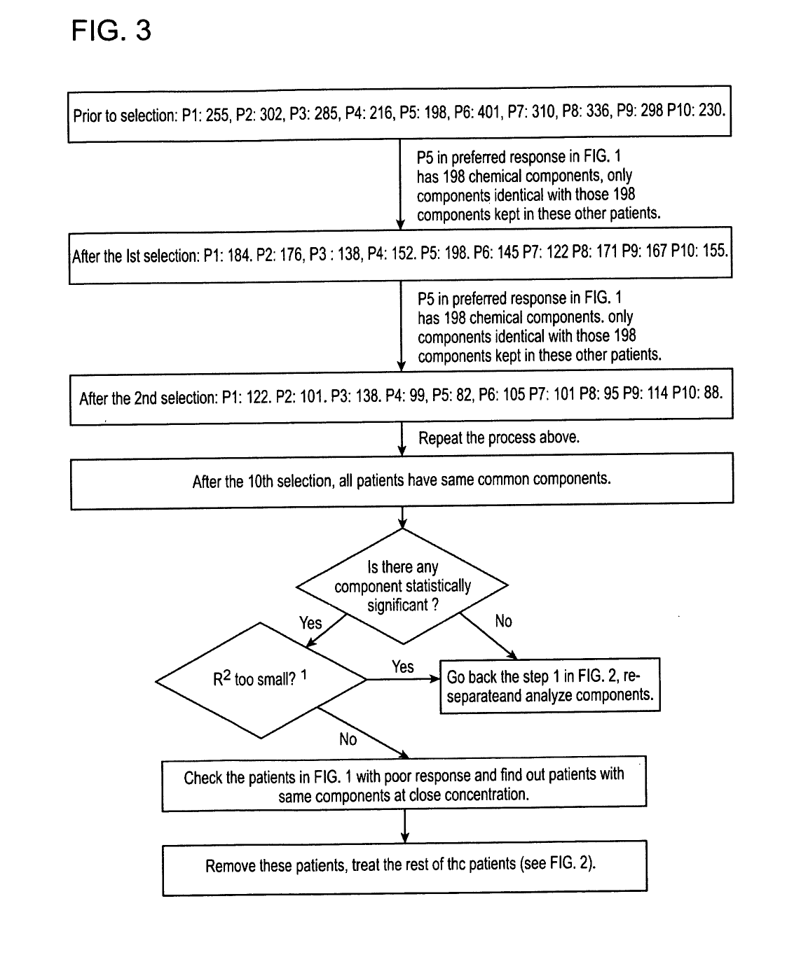

There are numerous ways to analyze the chemical compositions, such as high pressure liquid chromatography (HPLC), GC (GC-MS), liquid chromatography-mass spectrometry (LCMS), liquid chromatography mass spectrometry (LC-MS-MS), TMC fingerprint, and so on. There are numerous chemical compositions in each individual, by analysis of blood specimens from the individual, common components among patients with preferred response should be detected, with a computer-aided programs as explained in FIG. 3. Referring to FIG. 3 assuming 10 patients (p1 to p10) with the preferred response in FIG. 1, every patient has different components with chemical analyses, for example: p1 has 255, P2 has 302, etc. One can find out common components with the logic explained in FIG. 3. Regarding what explained in FIG. 3 If R2 too small, that means some important components lost, go back the step 1 in FIG. 2 and try to discover lost components. Since the patients with same components at close concentration, there is no reason to subject them in the test.

Statistical analysis could be applied and non-statistically significant compounds can be removed, R-Squared could used to determine how much remaining compounds to explain the efficacy as explained in FIG. 2. Regarding what explained in FIG. 2 the components are safe and effective when together with herbals in some patients, the concentration could be found out in those patients. In 6a, the patients in poor response in FIG. 1 are treated with components together with herbals, whereas in 6b the patients in poor response in FIG. 1 are treated with components only, the purpose is to eliminate non-essential components.

Logistic regression could be applied to remove non-significant compositions with a binary data as dependent variable, ANCOVA could be applied for a linear dependent variable, and survival analysis will be used with for long-term study. Clinical improvement will be dependent variable, and the chemical compositions will be dependent variables. Non-significant independent variables will be removed if p-value>=0.05.

There are still statistically significant compounds after the analysis, effort will be made to identify these compounds through chemical database, If structures of those compounds are known, and could be available, they can be used in next step for in vitro and in vivo studies, if not; those compounds could be purified for further study.

Safety of these compounds are available in clinical application during treatments, and hundred year experience, the range can be discovered by studying blood samples from different time points. These purified or synthetic compounds could be used in animal studies, if they are safe, and they can be used in treatment of patients independently or mixed together with the original concoction. Outcome can further prove these compounds safe and effective as explained in FIG. 2.

To eliminate useless ingredients in herbals, the synthesized compound can be used alone to treat the patients. If some patients have preferred results, these compounds are proved to effective and can be used alone clinically.

Analysis of the components of each original herbal and mixture of these herbals can be performed, and the results could be compared with other compounds i chemical databases to see if they are structurally known. Active compound may be present in the original herbals, or the mixed herbals. If these are not present in these herbals, they might be in vivo metabolites.

### MODE FOR INVENTION

Case 1: Discovery effective components to treat hypertension from herbals.

A TCM doctor treats hypertension with concoction composed by A, B, C, D, E herbals, twenty patients were treated. Initially, all patients have high blood pressure and without other disease. These patients are treated and come back to the clinic once every three days. After two weeks, blood pressure among ten patients becomes normal; there is no change in blood pressure in the other ten patients. Blood collection is done at times at 0, 0.5, 1, 2, 4, 8, 12, 24 hours after the treatment.

Logistic stepwise regression are applied with finding that A, B, C, E are statistically significant herbs, R-Squared of those herbals=0.81 with these herbals, that doctors remove D herbal from the concoction, and continued to treat patients. This result is confirmed in the treatment of patients with other doctors.

By LC-MS-MS analysis of blood samples from those patients with good responses to the treatment, there are hundreds chemical components each patient, only four identical ingredients W, X, Y, Z. are detected. Logistic stepwise regression was applied with the W, Y, Z statistically significant compounds, R-Squared for these 4 components=0.84.

Database are searched, Y is a known compound and can be synthesized, and Z is unknown compound. W is a compound in herbal A, Y is a compound in the B, Z is neither present in A, B, C, E herbal, nor present in the mixture of A, B, C, E, therefore, Z must a metabolite of either A, or B, or C, or E, a vivo metabolite vivo.

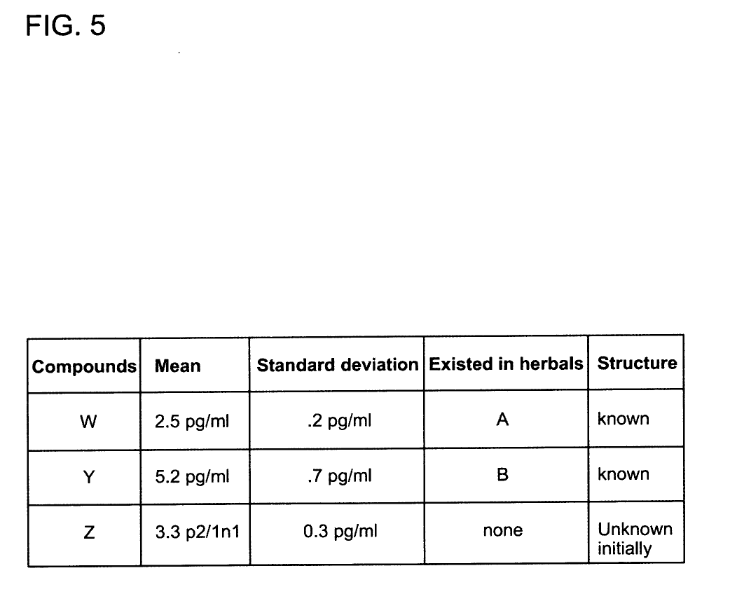

The structure of Z is later known with study and can be synthesized. Chemical analysis showed that the highest concentration of W, Y, and Z in each patient as explained in the table in FIG. 5, all the numbers in this table are hypothetical ones.

Further analysis are done by in the same method in 10 patients who did not show good response to check if there exist W, Y, Z compounds, two patients (P1, P2) contain W, Y with concentration close to these patients with a marked effect, but there is no Z compound, wherein three patients (P3, P4, P5) have no W, Y, Z, the blood sample from patients (P6, P7) have W, Y, Z, but their concentrations are very low, the other three patients (P8, P9, P10) have W, Y, Z in their blood, and concentrations are close to the patients with good response. The compounds W, Y, Z are synthesized and are used to treat 7 patients (P1 to P7) every day, their blood pressure are checked after 2 weeks.

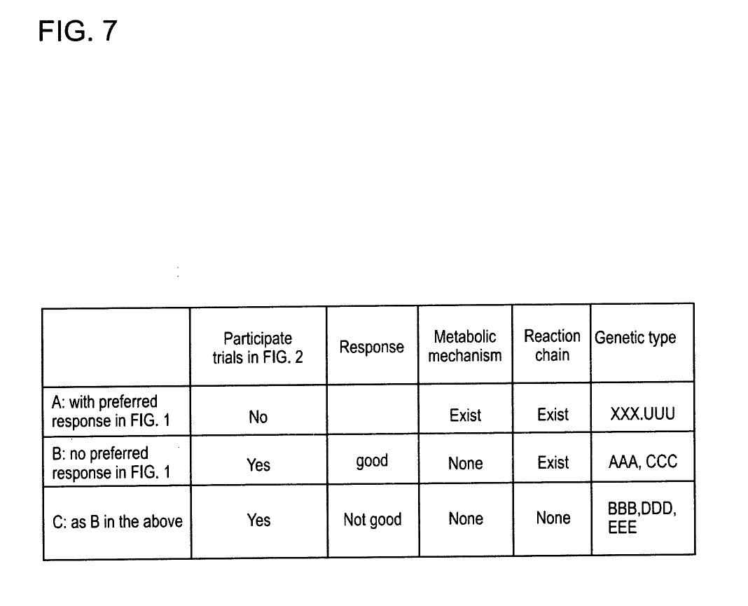

By gene analysis there show 5 gene types among P1-P10: AAA, BBB, CCC, DDD, EEE Type AAA patients only need Z compound for the treatment, the type CCC need all W, Y, Z compounds for the treatment, while types BBB, DDD, EEE patients are not treatable with any or all these compounds. The results are shown in the table in FIG. 7:

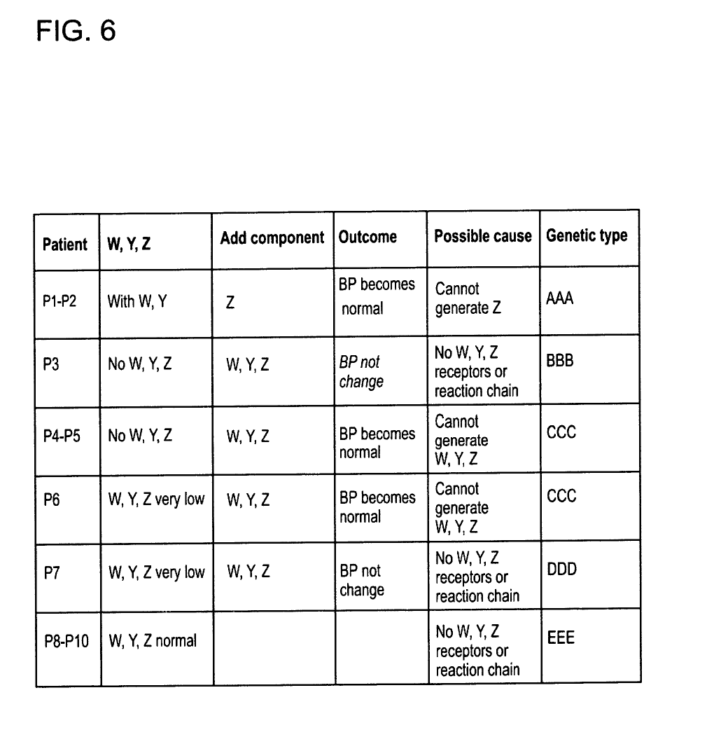

The genotype for ten patients with a good response is HHH. In the future treatment, patient's genotype should be checked first; if the genotype is AAA or CCC, or HHH, the compound W, Y, Z can be applied for the treatment, patients with genotype BBB, DDD, EEE should not be treated with these compounds as explained in FIG. 3, in the table in FIG. 6 and in the table in FIG. 7.

### EMBODIMENT 2

Discovery anti-bacterial components in herbals, Doctors use herbals to treat patients with E. coli septicemia, there are ten patients treated with the first concoction. Blood collection are done at time points 0, 0.5, 1, 2, 4, 8, 12, and 24 hours after patients take the concoction. In vitro collection tests are done, and the result is as follows:

At 0 hour, the mean of the colony was 52 with standard deviation (S.D) 11; after 24 hours of the treatment, the number of the colony was 222 with S.D. 35. the first concoction in-effective. The result demonstrates the concoction ineffective.

Then doctors use the 2nd concoction to treat same patients. Blood collection are done at the same time points. In vitro collection tests are done, and the result is as follows:

At 0 hour, the mean was 522 with S.D 41; after 24 hours of the treatment, the mean was 15 with S.D. 3. the 2nd concoction is effective.

Based on these results, the second prescription is proved to be effective. Analysis of blood samples of patients with the preferred effect, each patient has many chemical components, only ingredients C1-C4 present in all patients.

Analysis of patient's blood samples treated with concoction 1, C1-C2 were found, chemical databases shows that C3-C4 are are structurally found is known and can be synthesized and commercial available.

The C3-C4 are tested on the in vitro Petri-dish and found that they have prominent activities for killing bacteria. The peak concentration can be determined by measuring the blood samples collected at different times from these patients, the highest concentration are obtained by measuring blood samples at different times, and proper dosage for C1-C4 can be decided. There is no observation of side effects in these patients, and no reports of side effects from hundred years practice with these herbals

Based on concentrations in those patients at different time points, and vitro toxicity tests are done with C3-C4. After the pass, the volunteers for safety testing are performed. If the patients in the trial show preferred reaction and no apparent side-effect, they can be used to treat patients.

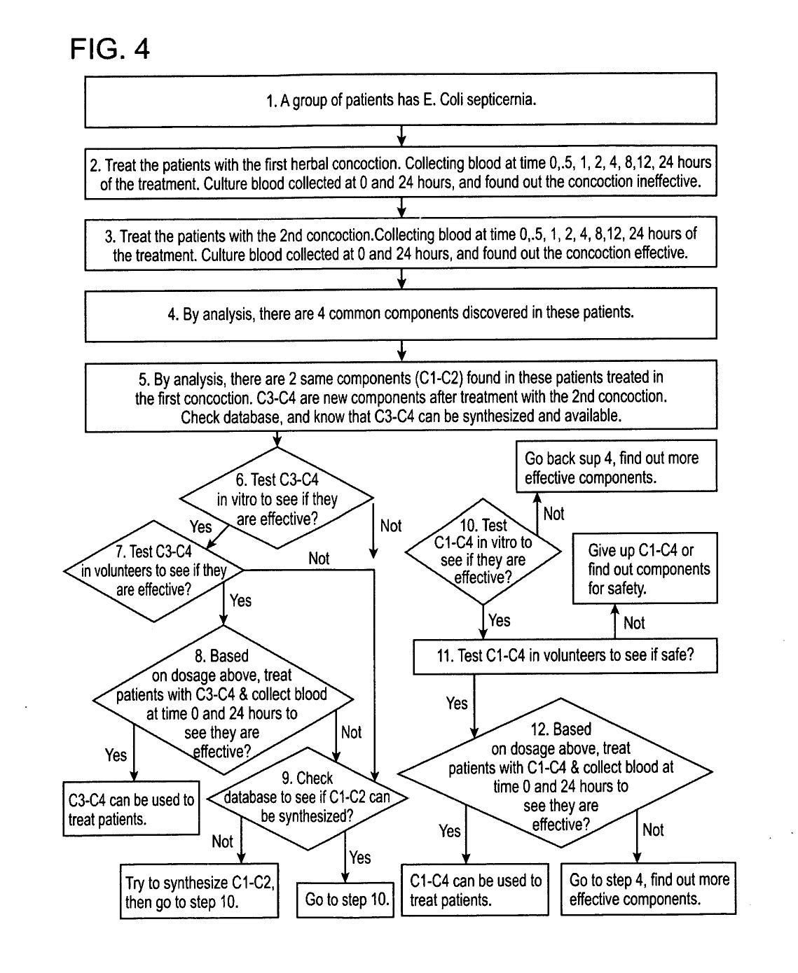

C3-C4 non-effective or with significant adverse effect, and chemical databases are recheck, the structures of C1-C2 are also known and are commercially available. The highest concentration are obtained by measuring blood samples at different times, and proper dosage for C1-C4 can be decided. Vitro validity tests are done with C1-C4 without issues. The volunteers for safety testing are performed, and there is no observation of side effects in these patients, and no reports of side effects from hundred years practice with these herbals. C1-C4 can be done in volunteers for safety and efficacy. The preferred reaction are observed, and no significant side effects seen, the C1-C4 drug can treat the disease, the whole process is shown in FIG. 4.

Note: If the structures on C1-C4 are unknown or they cannot be synthesized, they may be purified or tested. If they are effective, then the components can be analyzed and synthesized later.

The above cases are limited examples in order to illustrate the application of this patent not exhaustive, there may be other unlimited embodiments within the scope of the present invention.

Referring to FIG. 6 this is the table to show patient's response and their genetic types.

1. Those patients are hypothetical ones who have no preferred response with the first concoction.

2. The genetic types are hypothetical ones.

3. BP: blood pressure.

4. There is no reason to add W, Y, Z components, since these components exist in those patients within normal range.

If a patient produce preferred response to herbal treatment, some components must exist for the response, they could be different components existed in the herbs, or reaction components from different herbals, or metabolic compounds within human body. Patients with preferred response should have reaction mechanisms, such as receptors, chain reaction, enzyme subtypes, and so on. Depending on the patient's reaction to herbs, they can be divided into three categories: A, B, C.

Referring to FIG. 7 this is the table to show the classification of patients based on their response to herbals. Types A and B patients can be treated with by these herbals, whereas type C patients cannot be, as these patients lack necessary reaction chain for the herbals; patient types can be identified by scientific methods. Type C patients should be treated with these herbals. All information is hypothetical ones.

1. A method of identifying active ingredients of traditional Chinese medicine, including the steps of: setting up a website to allow providers to record patients' data including patients' condition, diagnosis and treatment through the website, wherein the providers are able to download said patients' data; performing statistical analysis on said data by means of ANOVA, ANCOVA, Logistic regression, time series, R-square, etc. to discover effective herbal combinations and eliminate non-critical herbs; analyzing the compositions of those herbals, by synthesizing or separating compounds within said compositions; conducting both internal and external tests to determine the active ingredients, and treating patients with pure effective compounds or together with combination of herbals; and effective compounds on patients for their medical conditions with or without approval by regulatory agencies.

2. The method of claim 1, wherein said website is set up to allow every practitioner to input practice information and conduct statistical analysis upon the data downloaded from the website, and the practitioners are able to make continuous improvements of diagnosis and treatment, repeatedly, and determine which herbs safe with positive effects on patients and which herbs safe but not effects on patients.

3. The method of claim 1, further comprising: analyzing patients' blood specimens with chemical analytical instruments, and finding the common composition of those patients with positive outcome via computer program.

4. The method of claim 1, further comprising: analyzing and removing the non-critical composition of the compounds through statistical methods, including one selected from ANOVA, ANCOVA, Logistic regression, time series, R-square, etc.

5. The method of claim 1, further comprising: analyzing blood specimens among those non-effective patients and finding out who has these common composition.

6. The method of claim 1, further comprising: determining whether these common compounds are effective with both internal and external tests by the differences between the diverse compounds included in patients with positive and negative outcomes.

7. The method of claim 1, further comprising: conducting both internal and external tests with purified compounds according to patients' biological characteristics.

8. The method of claim 1, further comprising: treating patients with discovered active compounds according to their different biological characteristics.

9. The method of claim 1, further comprising: treating patients by only the purified active compounds or to do so with combination of the purified with the original herbs.

10. The method of claim 1, wherein effective compounds can be used to treat patients for relevant medical conditions, after approved by regulatory agencies, or as patented materials without the approval, or as trade secrets.
